(19) United States
(12) Patent Application Publication
(10) Pub. No.: US 2006/0135911 A1
(43) Pub. Date: Jun. 22, 2006

(54) Temperature modulation of transdermal drug delivery

(76) Inventor: Aravindkumar Mittur, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/304,335

(22) Filed: Dec. 14, 2005

Related U.S. Application Data
(60) Provisional application No. 60/637,113, filed on Dec. 17, 2004.

(51) Int. Cl. A61F 7/12

(52) U.S. Cl. 604/113

(57) ABSTRACT

A trans-body-surface drug delivery device for the administration of at least one drug to an individual at a therapeutically effective rate. The device including a reservoir having at least one drug and a thermoeffector having a first surface that is controllable to at least one of heating and cooling the reservoir to affect passage rate of the drug through the body surface.

### CROSS REFERENCE TO RELATED U.S. APPLICATION DATA

The present application claims the benefit of provisional application 60/637,113 filed Dec. 17, 2004.

### FIELD OF THE INVENTION

This invention relates to the trans-body-surface drug delivery. In particular, the invention relates to transdermal drug delivery systems and methods using temperature changes to enhance drug delivery.

### BACKGROUND

The natural barrier function of the body surface, such as skin, presents a challenge to delivery therapeutics into circulation. Devices have been invented to provide transdermal delivery of drugs. Transdermal drug delivery can generally be considered to belong to one of two groups: transport by a “passive” mechanism or by an “active” transport mechanism. In the former, such as fentanyl transdermal systems available from Janssen Pharmaceuticals and other drug delivery skin patches, the drug is incorporated in a solid matrix, a reservoir with rate-controlling membrane, and/or an adhesive system.

Passive transdermal drug delivery offers many advantages, such as ease of use, little or no pain at use, disposability, good control of drug delivery and avoidance of hepatic first-pass metabolism. Most passive transdermal delivery systems are not capable of delivering drugs under a specific profile, such as by ‘on-off’ mode, pulsatile mode, etc. Consequently, a number of alternatives have been proposed where the flux of the drug(s) is driven by various forms of energy. Some examples include the use of iontophoresis, ultrasound, electroporation, heat and microneedles. These are considered to be “active” delivery systems.

Iontophoresis, for example, is an “active” delivery technique that transports solubilized drugs across the skin by an electrical current. The feasibility of this mechanism is constrained by the principles of thermodynamics and electrochemistry. A significant advantage of active transdermal technologies is that the timing and profile of drug delivery can be controlled, so that doses may be automatically controlled on a pre-determined schedule or self-delivered by the patient based on need. However, for such devices, there is still a lack of adequate delivery control over a suitable period of time. Some approaches to active transdermal delivery involve increasing skin permeability by heating the skin, thereby allowing drugs to permeate the skin more effectively and efficiently than otherwise without heating. The application of thermal energy aids skin permeation by several mechanisms, including: enhanced permeability of skin; increase in systemic circulation and dilation of blood vessels; and enhanced release of the drug from local skin tissue into systemic circulation.

However, using increased temperature to increase microcirculation and drug solubility also presents certain challenges. A prolonged period of heat application may slightly decrease the barrier property of the skin, which may result in increased irritation as well as uncontrolled levels of drug in the skin and systemic circulation. Certain patch-like heating devices have been disclosed in which heat is chemically generated by oxidation that is modulated by varying the exposure of the patch surface to oxygen. Such a device, when placed on top of a passive transdermal patch, is reported to increase the temperature of skin and subsequently the absorption of a drug being administered by the patch. Some have proposed to include heating elements such as chemical, electrical and infra-red mechanisms. Yet others have proposed to use short and rapid bursts of thermal energy to create pores in the surface of the skin, which may be done by including an external device that provides a heat source to metallic filaments embedded in the patch. Exemplary patents that are related to using thermal energy to effect drug delivery include U.S. Pat. Nos. 5,226,902 and 6,488,959.

ALZA Corporation has published studies to demonstrate that transdermal flux of fentanyl from patches increases with body temperature (J. Pain Symptom Manage 7: S17-S26, 1992). The data showed that, with the assumption the diffusion rate of drug from the system remains unchanged, increasing the temperature by 3° C. can increase the maximal concentration of fentanyl in circulation by 25% during peak delivery. Further, there has been evidence that the transport of FITC-Dextran (10 kDa) across pig epidermis is markedly enhanced at temperatures over 40° C. relative to those observed at temperatures below 37° C., and that the temperature induced increase in passive transport and in electrogenic transport are additive (Narasimha Murthy et al, J. Pharm. Sci. 93:908-915, 2004).

Thus, increasing temperature seems to be effective in enhancing drug delivery. Yet, mechanisms that can control the rate and amount of drug being delivered are lacking. Thus, there is a need for systems and techniques that can provide better control in such temperature assisted drug delivery.

### SUMMARY

The present invention provides devices and methods for trans-body-surface administration of at least one drug to an individual at a therapeutically effective rate, by using temperature to control the flux of the drug. In one aspect, the invention uses controlled heating and cooling of a drug reservoir to affect passage rate of the drug. An effective way to control the delivery rate of the drug is to control the amount of drug composition that is available to the body surface.

In an aspect of the invention, the amount of drug composition made available to the body surface is controlled by reversibly producing a temperature change to a drug reservoir proximate to the skin.

In an aspect of the invention, a device and method are provided for the administration of a liquid to a body surface by controlling both heating and cooling of a matrix (or carrier) to cause the matrix to change volume, thereby controlling the amount of liquid being made available to the body surface.

In an aspect of the invention, a device and method are provided for the transdermal delivery of a drug. The transdermal flux of the drug can be modulated by reversibly heating or cooling the matrix. In an embodiment, a liquid contains a drug in a matrix and a Peltier device having a first surface is reversibly controllable to both heat and cool at different times to cause the matrix to swell and shrink, thereby controlling the amount of drug passing through the surface. In the past, conventional transdermal systems are normally limited to very potent (low dose), lipid-soluble drugs with a molecular weight of less than 500 Daltons. The present invention provides a way to further improve the efficiency of transdermal flux of drugs and allow a significantly broader range and type of drugs to be delivered at higher dose levels. To this end, the present invention increases the transport of the drug(s) trans-body-surface by making the drug composition more available to the body surface and by increasing the kinetics of transport due to a higher temperature. The present invention provides finer control of drug delivery over the prior art and enables therapeutically effective drug delivery. In one aspect of the present invention, reversible temperature modulation enabling the reversible swelling and de-swelling of thermosensitive hydrogels, making it possible to control the release of an embedded drug or mixture of drugs. In other instances, the ability to aid and reverse transdermal flux may also allow for a greater dynamic range of dose of compounds that may otherwise be limited by flux across the skin.

Thermosensitive hydrogels swell or shrink in response to changes in temperature. For example, in certain embodiments, an active drug that is incorporated in such a hydrogel will be released when the hydrogel shrinks in response to temperature change, e.g. by heating. Conversely, when such a hydrogel is subsequently cooled to an appropriate temperature at which it re-swells, residual drug in the chamber will be re-incorporated back into the hydrogel. Thus, the availability and/or release of the drug from the hydrogel matrix can be easily controlled.

The device of the present invention can be used for assisting passive or active trans-body-surface drug delivery. When coupled with electrotransport based (active) trans-body-surface delivery technology or passive trans-body-surface delivery technology, the present invention affords the following advantages:
a. Recurring pulsatile delivery of drugs by reversible thermal activation of matrix.
b. Provides an “on-demand” mechanism to control drug release.
c. Enhance control of electrogenic trans-body-surface flux of drugs from conventional trans-body-surface delivery.
d. Minimize potential formation of drug depot in the skin and thus rapidly increase drug concentrations in the systemic circulation.
e. Enhance stability of drugs in the delivery device by trapping moisture and releasing it only when required.
f. Improve electrotransport device by isolating drug reservoir and controlling amounts of ‘free fluid’ by ‘just-in-time’ release from reservoir.
g. Potentially minimize drug abuse by stable incorporation in a hydrogel matrix at sub-optimal temperatures.
h. Deliver small water-soluble drugs as well as macromolecular drugs and vaccines at doses not achieved by traditional transdermal delivery.

### BRIEF DESCRIPTION OF THE DRAWINGS

The present invention is illustrated by way of example in embodiments and not limitation in the figures of the accompanying drawings in which like references indicate similar elements.

FIG. 1 is a schematic illustration with a sectional view of an embodiment of the thermally controlled drug delivery device of the present invention.

FIG. 2 is a schematic illustration of a plan view of portion of the embodiment of FIG. 1.

FIG. 3 is an illustration showing a portion of a thermoeffector of an embodiment of the thermally controlled drug delivery device of the present invention.

FIG. 4 is a schematic illustration with a sectional view of an embodiment of an iontophoretic drug delivery system in portion according to the present invention.

FIG. 5 is an isometric exploded view of an electrotransport drug delivery device that can be adapted to have temperature control according to the present invention.

FIG. 6 is a cross-sectional view of one embodiment of a transdermal therapeutic drug delivery device that may be used in accordance with the present invention.

FIG. 7 is a cross-sectional view of another embodiment of a transdermal therapeutic drug delivery device that may be used in accordance with the present invention.

FIG. 8 is a cross-sectional view of yet another embodiment of a transdermal therapeutic drug delivery device that may be used in accordance with this invention.

### DETAILED DESCRIPTION

In describing the present invention, the following terms are intended to be defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation.

“Pharmaceutical agent” is to be construed in its broadest sense to mean any material that is intended to produce some biological, beneficial, therapeutic, diagnostic or other intended effect, such as relief of pain and contraception. Unless specified differently in context, as used herein, “drug” and “pharmaceutical agent” are used interchangeably herein.

As used herein, the term “therapeutically effective” refers to the amount of drug or the rate of drug administration needed to effect the desired therapeutic result. As used herein, the term “permeation enhancement” intends an increase in the permeability of skin to a drug in the presence of a permeation enhancer as compared to permeability of skin to the drug in the absence of a permeation enhancer.

The term “thermoeffector” refers to an electric device that has a surface that can be electrically activated to effect a temperature change to a material in thermoconductive contact therewith by heat conduction.

The term “intact body surface” refers to a body surface, such as intact skin surface, that does not have wounds or injuries, and has not been punctured by sharp objects.

The term “actively reversibly heating” refers to heating that can be reversed into cooling by changing a heating surface to a cooling surface to transfer energy from one location to another, not merely by dissipation of heat to the environment in an uncontrolled fashion.

The present invention provides novel devices and technique for delivery of drug(s) trans-body surface to a patient. Heating and cooling is used to control the delivery of the drug(s) through a body-surface of the patient. The body surface can include intact skin and mucosa. The body surface, for example, may be on the external of a body, such as the back, or in the buccal or rectal locations, or even within the channel of the ear, on the eyeball or on the side of the eyelid facing the eye. In the following description of illustrative embodiments, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

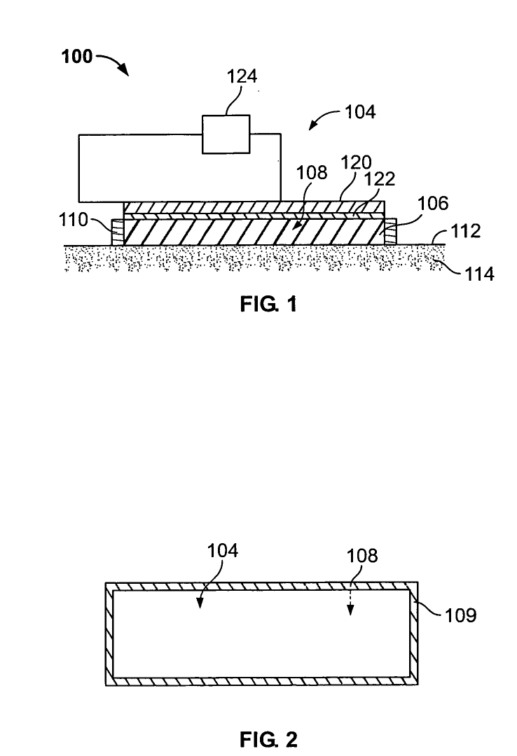

In one aspect, the present invention utilizes a device schematically shown in FIG. 1 for controlling transdermal drug delivery. A transdermal drug delivery device 100 includes a Peltier device 104 being in thermoconductively contact with a matrix 106 in a drug reservoir 108 confined within reservoir walls 110. The matrix is suitable for affixing to a body surface 112 of tissue (such as intact skin) 114 for drug delivery. The Peltier device 104 includes a thermoeffector 120, which is plate-shaped having a surface (not shown in FIG. 1) suitable for intimate thermoconductive contact through a thermoconductive seal 122 to the matrix 106. Preferably the thermocunductive seal 122 is thin and thermally conductive to provide efficient heat transfer between the matrix 106 and the thermoeffector 120. It is contemplated that the drug reservoir 108 can contact directly the thermoeffector 120 for direct thermal conduction.

The Peltier device 104 includes electronic circuitry 124 for controlling the thermal operation of the Peltier device. A Peltier device of an appropriate size to adequately cover the drug reservoir for application to a particular body surface area under treatment can be used. The Pelteir device can vary in size depending on the treatment need. It can have a surface area for conductive contact with the drug reservoir from, for example, a few mm² to hundreds of mm². For transdermal drug delivery, the surface area is preferably between 5 to 100 mm², preferably between 10 to 50 mm². The thickness of the plate-shaped thermoeffector 120 can be a few mm, preferably is less than 4 mm for ease of use on the skin, more preferably between 1-4 mm. Because a thin device is generally desired for body surface application, generally a thin thermoeffector is desired. Often, the thermoeffector 120 has a thickness less than that of the drug reservoir. Peltier devices of the right size (capacity, size, and thermal output) can be purchased from a number of commercial sources. Alternatively, they can be manufactured to suit custom specifications.

Due to the solid-state nature of a Peltier device, it is possible to precisely, rapidly, uniformly and reversibly control the temperature of a drug reservoir. For example, the release of a drug incorporated in a hydrogel that is highly viscous at 15-37° C. may be facilitated by rapidly raising its temperature to a pre-defined degree. The release of drug from the matrix may subsequently be reversed by cooling the reservoir. On the face of the Peltier device facing away from the drug reservoir, optionally a thermal sink can be provided to receive heat when the drug reservoir is being cooled and to provide heat when the drug reservoir is being heated.

Although the present invention is not limited by a scientific theory, a Peltier device is a thermoelectric device and acts as a heat pump that can work without moving parts, fluids or gases. It has cold and hot junctions. At the cold junction, where the temperature falls, energy is absorbed by electrons as they pass from a low energy level in the p-type semiconductor element to a higher energy level in the n-type semiconductor element. At the hot junction, where temperature increases, i.e. changes in the opposite direction to that of the cooling junction, energy is transferred to the environment (which may be tissue or heat sink) as electrons move from a high energy level element (n-type) to a lower energy level element (p-type). Thermoelectric devices have been used in the past for cooling, for example, as disclosed in U.S. Pat. Nos. 6,492,585; 6,613,602; 5,448,109; and 6,345,507, the description of which relating to thermoelectric devices and the control and use thereof are incorporated by reference in their entireties.

Typically, in a Peltier device, thermoelectric heating or cooling couples are made from semiconductor material, typically bismuth telluride, although other semiconductor materials in different arrangement can be used, for example, bismuth chalcogenide material made from bismuth-telurium-antimony and cobalt antimony materials. The semiconductor material, such as the bismuth telluride, is doped to create either an excess (n-type) or deficiency (p-type) of electrons. The p-type and n-type materials are fashioned into to thermoelectric element, typically as cube or rectangle-shape pieces (sometimes called “couples”) and arranged in pairs of n-type and p-type elements in an array in a thermoelectric module. The couples are electrically connected in the array, typically in series for efficiency of construction, and electrically communicate with circuitry that supplies current to control the heating and cooling of the couples. The energy to move the electrons through the system is supplied by a power supply. The conductors that connect the couples can be arranged to be in generally planar fashion to provide a surface to fit reasonably well with the surface of the material to be heated or cooled. As current is passed through the array, heat absorbed at the cold junction is pumped to the hot junction at a rate proportional to current passing through the circuit and the number of couples, thereby effecting heating or cooling on the surface of the material whose temperature is to be adjusted.

To maintain electrical function, the conductors and circuitry are generally insulated in a Peltier device. Typically, in industrial applications, the semiconductor couples and the conductors connecting the p-type cubes to the n-type cubes are sealed between ceramic plates in the form of a sandwich. In the present invention, for application of body tissue surface, it is understood that the semiconductor material and conductors can be sandwiched between alternative insulation materials such as polymeric materials that provides the thermal and mechanical integrity within the operational temperature range of the drug delivery device. Furthermore, the edges of the Peltier devices are to be sealed to prevent moisture from reaching the semiconductor materials and the conductors. Materials for forming the insulation plates for sandwiching in the semiconductors and conductors include, for example, ceramic materials such as aluminum oxide, aluminum nitride, and beryllium oxide. Further, it is contemplated that within the temperature of application of the thermoelectric device on a physiological body surface, polymeric materials can be used for making the insulation plates. Applicable polymeric materials include halogenated material such as poly(tetrafluroethylene), poly(vinylidene fluoride), fluroalkylsiloxane elastomers, polyesters such as poly (ethylene terephthalate) and poly (4,4′-isopropylidine-diphenyl barbonate); polyethers such as polyformaldehyde and poly(2,6-xylenol), polyimides, poly siloxanes, polyalkylenes such as polyethylene and polypropylene; polysulfones; copolymers such as polyvinyl and polyolefin copolymers, including poly(acrylonitrile-vutadine-styrene)copolymers, poly(vinyl chloride-acetate)copolymers; poly(methyl methacrylate); and the like.

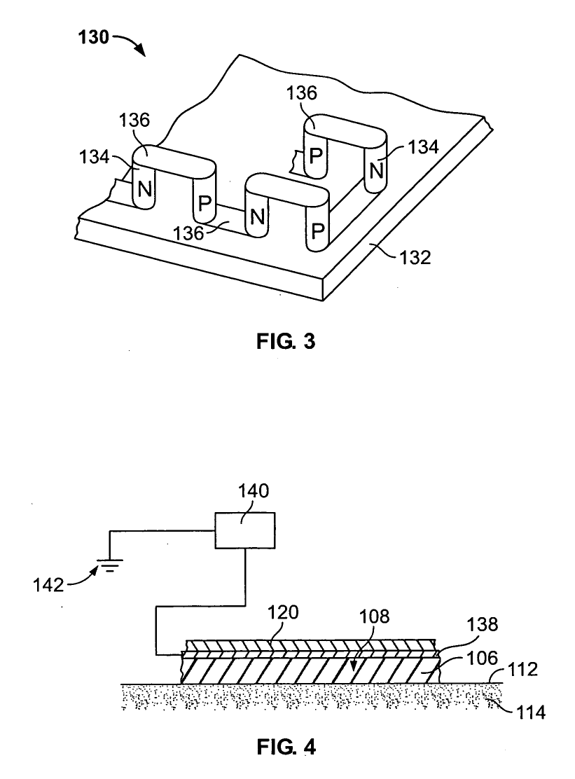

FIG. 3 illustrates a portion 130 of the thermoeffector 104 of a Peltier device that can be used in the present invention. The thermoeffector portion 130 includes a contacting plate 132 that can be thermoconductively sealed to the drug reservoir through a thermoconductive seal (not shown in FIG. 3). On the contacting plate 132 are n-type and p-type couples 134 that are connected via electrical conductors 136 in series. The ends of the couples 134 and the electrical conductors 136 are in thermal contact of the plate 132 for effective heat transfer. A second plate (not shown in FIG. 3 so as not to obscure details) contacts the conductors 136 on the side opposing to plate 132.

The Peltier device in the present invention is adapted for reversible hot-cold temperature modulation so as to control the delivery of drug from the drug reservoir. Modulation of heating and cooling is enabled by switching polarity of the applied voltage. Voltage will be reversed, for example, by the utilization of a standard sub-miniature relay, e.g. a “double-pole, double-throw” (DPDT) relay. Another alternative is using a metal oxide semiconductor field effect transistor (MOSFET) inverter to reverse the flow of direct current through the couples. Other electronic means (including programmable circuitries) for reversing current flow can be implemented. The heating and cooling cycles can be governed by a feedback mechanism, which can be used to control at a pre-defined temperature or thermal curve. The feedback regulator may include a temperature sensor for sensing a temperature that is being controlled. For example, in the case in which a hydrogel is used in the matrix, the control point for feedback regulation can be chosen around a temperature at which the gelation state of the hydrogel is to be modified.

The delivery system preferably contains a matrix that can change volume or its capacity to hold a fluid that contains the drug being delivered. A preferred material in the matrix is a stimuli-sensitive polymer hydrogel, which can swell or shrink (or deswell) in response to changes in the environmental conditions. Shape memory multiblockcopolymers of macrodiols are described in the literature (Langer, R., Nature 392 (suppl.): 5-10, 1998; Peppas, N. A. Curr Opin Colloid Interface Sci 2: 531-537, 1997. Hydrogels that change structurally with temperature changes such as copolymers of (meth)acrylic acid, acrylamide and N-isopropyl acrylamide, water-soluble synthetic polymers crosslinked with molecules of biological origin, such as oligopeptides and oligodeoxyribonucleotides, or with intact native proteins as disclosed in the following references can also be used in the matrix for delivery of drugs according to the present invention. The description of the polymers and technique for causing changes to their structure and shape of the following references are incorporated by reference in their entireties: US Pat. No.5,226,902 (related to temperature sensitive hydrogels with polymers made from monomers such as N-isopropylacrylamide, N,N-diethylacrylamide, acryloylopiperidine, N-ethylmethacrylamide N-n-propylacrylamide and N-(3′-methoxypropyl)acylamide); Yoshida et al., Adv Drug Deliv Rev 11:85-108, 1993 (related to pH-sensitive hydrogels such as those made from acrylic acid or aminoethyl methacrylate; electro-sensitive hydrogels such as those made by crosslinking poly(2-acrylamido-2-methylpropane sufonic acid) and temperature-responsive gels, such as those made from Poly(N-isopropylacrylamide) (i.e. poly(NIPAAm); Li and E'Emanuelle, Int. J. Pharmaceutics 267: 27-34, 2003 (related to thermoresponsive Poly(N-isopropylacrylamide) (i.e. poly(NIPAAm)hydrogels; Dinarvand and D'Emanuele, J. Control. Release 36,:221-227, 1995 (related to use of thermaresponsive hydrogels, such as those made from Poly(N-isopropylacrylamide) for on-off release of molecules). Polymers suitable for incorporation in the matrix to effect temperature responsive in structure and shape for drug delivery include poly(N-isopropylacrylamide)homopolymer, poly(N-isopropylacrylamide)acrylamide copolymer, copolymer of poly(N-isopropylacrylamide) containing silane monomers such as [3-(methacryloyloxy)propyl]trimethoxysilane, [2-(methacryloyloxy)ethoxy]-trimethylsilane and/or methacryloyloxy)trimethylsilane, copolymer of poly(hydroxypropyl methacrylamide) and dicarboxymethylaminopropyl methacrylamide with protein moieties, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene)derivatives.

It is noted that a single type of polymer or a blend of different polymers can be used so long as the matrix can functionally swell and shrink in response to temperature change for releasing and absorbing drug composition. Preferably, polymers formed from NIPAAm are used. Preferably, the polymeric portion of the matrix includes about 20 to 100% by weight of polymerized NIPAAm, preferably 50 to 100%, more preferably about 80% and above, more preferably about 95% and above, even more preferably about 100% by weight of polymerized NIPAAm (i.e. formed from NIPAAm monomer). Further, the polymeric portion of the matrix includes about 20 to 100% by weight of poly(NIPAAm), preferably 50 to 100% by weight of Poly(NIPAAm), more preferably about 80% Poly(NIPAAm) and above, more preferably about 95% Poly(NIPAAm) and above. Even more preferably, the polymeric portion of the matrix consists essentially of poly(NIPAAm)homopolymer.

By selection of the polymer to be included in the matrix, thermally sensitive hydrogel delivery systems can exhibit both negative controlled release, in which drug delivery is halted at temperatures above the volume phase transition temperature (VPTT), and positive controlled drug delivery, in which the release rate of a drug increases at temperatures above the VPTT. Many polymer solutions exhibit a Lower Critical Solution Temperature (LCST, i.e. volume phase transition temp), below which they exist in a hydrophilic, soluble state and above which the polymer chains become hydrophobic and precipitate from solution. At sufficient concentrations of the polymer, this transitions the fluid into a gel. Thermosensitive (or thermoresponsive) polymer gels shrink or swell with changes in temperature. For example, as the temperature rises above a critical value (LCST), the gel collapses, expelling liquid (e.g. drug solution), and thus shrinking in volume. The swelling behavior is reversible when lowering the temperature below the LCST. Polymer gels can either be physically or chemically associated, and the nature of the sol-gel transition is impacted by competing interactions such as ionic interaction, hydrophobic interaction, van der Waals force, and hydrogen bonding, that are functions of both the gel composition as well as the gel's aqueous environment. In Table 1 below, a range of transition temperatures is provided since transition temperature of polymeric hydrogels can be controlled by various factors such as hydrophilicity, pH, feed ratio and/or concentration of co-monomers incorporated. The transition temperature of some hydrogels can further be modified by inclusion of elemental particles, varying the concentration of crosslinkers, dithiotreitol, etc. Most hydrogels show negative or “normal” temperature sensitivity, i.e. they tend to take up water and swell at temperatures below the phase transition temperature. However, some hydrogels exhibit “reverse” thermogelation (i.e. form gels at elevated temperatures). Thus, such thermosensitivity or thermoresponsiveness is different from the general common place thermal expansion and contraction due to atomic vibrational energy change as a function of temperature change.

Table 1 lists exemplary polymers that can be prepared to exhibit temperature sensitivity. Poly(hydroxypropyl methacrylamide) can be modified by techniques such as partial esterification, e.g. with cinnamic acid, to result in copolymers with an LCST that can be adjusted over the full aqueous temperature range (A. Laschewsky, E. D. Rekai\ddot{}, E. Wischerhoff, Polym. Prepr. Am. Chem. Soc. Div. Polym. Chem. 40, 189, 1999). Such copolymers of poly(hydroxypropyl methacrylamide may be chosen to provide reversible swelling and shrinking for delivery of drug solutions.

| Polymer | LCST (° C.) | Type |
|---|---|---|
| poly(N-isopropylacrylamide) | 32 | Normal |
| poly(N-isopropylacrylamide)acrylamide | 32-65 | Normal |
| copolymer | | |
| poly(hydroxypropyl methacrylamide)copolymer | 25-65 | Normal |
| dicarboxymethylaminopropyl methacrylamide | 35-45 | Normal |
| xyloglucan | ˜40 | Normal |
| ethyl(hydroxyethyl)cellulose | 40 | Normal |
| ethyl(hydroxyethyl)cellulose + sodium | 32-40 | Reverse |
| lauryl sulfate | | |
| Hydroxypropyl methylcellulose | ˜40 | Reverse |
| Poly(vinyl methyl ether) | 37-40 | Normal |
| poly(ethylene oxide)/D,L-lactic acid-co- | 20-60 | Normal |
| glycolic acid (10-30 wt %) | | |
| Poloxamer 407 or Pluronic F127 | 20-45 | Normal |
| (8-16 wt %) | | |
| poly(acrylic acid)-g-Poloxamer | 4-37 | Normal |
| (0.5-3% w/v) | | |
| chitosan and polyol salts (with varying | 37-50 | Reverse |
| degree of deacetylation) | | |
| poly(silamine) | ˜37 | Normal |
| poly(organophosphazene) | 25-98 | Normal |

Poly(N-isopropylacrylamide) (poly(NIPAAm)) and its copolymers are particularly suitable for reversible delivery control in the present invention. Crosslinked poly(NIPAAm) hydrogel exhibits a volume phase transition temperature (VPTT) at approximately 32° C. in aqueous media due to the hydrophilic-hydrophobic balance of its constituent polymer chains and directly related to the lower critical solution temperature phenomenon exhibited by linear poly(NIPAAm) in aqueous solution. NIPAAm-co-AAm hydrogels can have a LCST ranging from 32-65° C., depending on the amount of AAm included in the copolymer. A copolymer hydrogel consisting of 95% NIPAAm and 5% AAm has a LCST of approximately 40° C. (J. H. Priest, et al. Reversible Polymer Gels and Related Systems 350:255-264 (1987); and L. C. Dong et al. Reversible Polymer Gels and Related Systems 350:236-244 (1987)). Hence, such a copolymer hydrogel is suitable for use in applications where it is desired to cause collapse of the hydrogel at temperatures only slightly above the normal core temperature of the human body.

Depending on the specific gel used, the change in volume of hydrogel around the LCST could be dramatic, with a large volume change within a small temperature range. However, the response times of drug release from a thermosensitive hydrogel is predictable and somewhat gradual rather than an “instantaneous” collapse. The rate of volume change is based on a number of factors, including conductivity, state of swelling or deswelling, etc. The response to temperature change over the LCST (e.g. 32 ° C. in some cases) can be gradual and predictable for the release of drug and can be determined experimentally. Some examples of liquid release of hydrogel with LCST are published by Lee and Yuan, J. Appl. Polym. Sci. 84: 2523-2532, 2002, which is incorporated by reference herein.

Although the extent of swelling of a hydrogel can vary, depending on factors such as the specific hydrogel composition, duration of stimuli, the number of swell/shrink cycles, type of initiator and type of crosslinker, the capacity of such a hydrogel with LCST is typically large. If swelling (q) is calculated as a mass ratio of the fully hydrated weight (Wh) to the dry weight (Wi) of the sample, i.e. q =Wh/Wi, the amount of swelling can be determined experimentally. The design of the temperature controllable drug delivery of the present invention can be implemented.

The thermosensitive polymers of the present invention can swell and shrink with temperature to result in therapeutically significant amount of drug solution release and absorption. Generally, the thermosensitive polymers can result in 50% to 2000%, preferably 100% to 1000% change in volume on swelling over a 15° C. temperature change around a base temperature of about 30° C. In other words, a hydrogel with a 100% change in volume on swelling absorbs an amount of aqueous material equal in weight to the dry weight of the hydrogel. The swelling and shrinking of the hydrogel due to the thermosensitive polymers is thus significantly larger than volume changes due to ordinary thermal contraction or expansion.

During use, the matrix in the reservoir may be activated by the thermoeffector to release 2-80% of the liquid (drug solution) held in the matrix. However, for maintaining better contact by the matrix to the skin and to the thermoeffector to effect temperature change, 5-50%, preferably 10-30% of the liquid may be released from the matrix during a period of use by a patient.

Generally, poly(NIPAAm) can be prepared by cross-linking N-isopropylacrylamide with crosslinkers, such as N,N′-methylene-bis-acrylamide (MBAAm) and accelerators such as N,N,N′,N′-tetramethylethylenediamine. The crosslinking reaction can be initiated using initiators such as ammonium persulphate and TEMED. Polymerization can be done in degassed distilled water with nitrogen bubbling to minimize oxygen presence. The poly(NIPAAm) can be made with varied degrees of crosslinking, for example, with a NIPAAm: MBAAm ratio of from 80 to 20, although ratios outside this range are possible. For example, 2.25 g of NIPAAM can be polymerized in 15 ml of degassed distilled water with crosslinker MBAAm in the amounts of 0.2% w/v, 0.4% w/v, and 0.6% w/v. After the polymerization, the synthesized hydrogels may be immersed in distilled water at room temperature for 48 hours and the water refreshed every several hours in order to allow the unreacted chemicals to leach out. A person skilled in the art will be able to adjust the crosslinking to make a polymer with the desired capacity for absorbing and releasing drug compositions. In another example, a hydrogel containing chitosan and glycerophosphate can be prepared by first preparing a solution of chitosan in deionized water and sterilized in an autoclave (121° C., 10 min). Then, a glycerophosphate solution can be prepared in deionized water and sterilized by filtration. The two solutions can be chilled in an ice bath for 15 min. The glycerophosphate solution is added dropwise to the chitosan solution with constant stirring and the resulting mixture is stirred for another 10 min under aseptic conditions.

Polymerized material can be formed into sheets, disks, and the like. In other ways, hydrogels can be prepared in a cylindrical or rectangular plastic tube in distilled water at 20° C. using TEMED (8.17 mol % based on monomer) and ammonium persulphate (1.91 mol %) as initiator and propagator, respectively. MBAAm (1.15 mol %) can be used as the crosslinker. Nitrogen can be bubbled through the solution for 15 min before the addition of the TEMED. The polymerization mixture can be left standing at room temperature for 1 hour to ensure that all the monomer has reacted. The prepared hydrogel block can be sliced into discs or rectangular slices and cleaned by repeatedly swelling in water followed by heating to approximately 50° C. They can then be filtered and dried in a vacuum oven at 50° C. for 48 hours.

Loading of small molecule drugs into hydrogels: The dried discs of a hydrogel can be loaded by sorption of an aqueous or ethanolic drug solution, followed by solvent removal in a dessicator at a requisite temperature to entrap the drug molecules. The requisite temperature would be chosen depending on the type and LCST of the thermosensitive hydrogel. For example, a hydrogel can be loaded with a drug solution at a temperature below the LCST of the polymer where it exists as a swelled or expanded form. A temperature switch above the LCST of the hydrogel will cause contraction or deswelling of the system, with the resulting magnitude and rate of contraction proportional to the extent of swelling prior to the temperature switch. In this example of a negative thermoresponsive hydrogel, increasing the temperature above the LCST will result in rapid contraction or shrinkage, thus releasing the drug solution from the hydrogel matrix. The loading content can be controlled by complete sorption of a known volume of drug solution for 48 hours (or requisite duration) in a suitable glass vial before drying out.

As an alternative, hydrogels can be loaded with drugs by placing them in contact for up to a week with 30 ml of buffer solution (25 mM HEPES and 50 mM NaCl) containing relevant concentrations of drugs.

Hydrogel formulations of chitosan and glycerophosphate containing drugs can be prepared by pouring the chitosan solution directly on the sterilized drug powder and stirring for 4 hours before mixing with the glycerophosphate solution as described above.

Dry copolymer hydrogel discs can also be loaded by immersion in 25 ml of a solution of the drug in acetone. The discs can be left in the drug solution to equilibrate for up to 3 days. Some hydrogels swell considerably in acetone and can thus provide the capability for achieving higher drug loading. The drug loaded discs can be removed from the solution and placed inside a vacuum flask. A controlled drying procedure can be used to minimize drug migration to the surface of the discs. Discs loaded with the drug can be dried under low vacuum for 3 hours at −20° C., 3 hours at −5° C., 6 hours at 5° C., and 12 h at 25° C. The drug loaded discs can then be dried in an oven (55° C.) for 12 hours.

Loading of macromolecular protein-based drugs into hydrogels: A drug loading solution can be prepared by first dissolving 2 g drug in 200 ml of phosphate-buffered solution (PBS, 0.1 M, pH 7.4). Before loading protein into hydrogels, each unswollen hydrogel can be vacuum dried for 1 day. Drug can then be loaded into the pre-fabricated dried unswollen hydrogel by equilibrium partition in a drug solution prepared as described above, i.e. by placing the vacuum dried hydrogel into a model protein such as bovine serum albumin (BSA) solution at 22° C. for a sufficient duration of time.

Any suitable trans-body-surface deliverable drug that can be held by the drug reservoir can be used in the present invention for delivery to the patient. It has been found that the following exemplary drugs work well with hydrogels for reversible control temperature modulation in delivery according to the present invention.
a. Lidocaine, Tetracaine (rapid onset and controlled levels)
b. Fentanyl, Buprenorphine (rapid onset and controlled levels)
c. Hydromorphone (acute post-operative and chronic pain)
d. Anti-Parkinon's disease agents such Apomorphine and Rotigotine (to supplement end-of-dose failures with traditional dopamine therapy, rapid amelioration of unpredictable motor complications such as bardykinesia and akinesia)
e. Photosensitizers used in photodynamic therapy, e.g. Photofrin II
f. RNAi-based therapeutics
g. Methylphenidate
h. Diclofenac (enhanced permeation)

Pharmaceutical agents, therapeutic agents, such as analgesics, that require rapid onset of action, as well as patient control on feedback, and those with a narrow therapeutic window would generally benefit from the present invention because of the enhanced flux and control. Other drugs that require further enhancement of transdermal flux for adequate bioavailability can also benefit from the present invention. Such drugs include therapeutic agents in all of the major areas, including, but not limited to, ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, antipyretics and anti-inflammatory agents, androgens, local and general anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretic, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinson's agents, antiplatelet agents, antiprogestins, antithyroid agents, antitussives, antiviral agents, atypical antidepressants, azaspirodecanediones, barbituates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, 5-hydroxytryptamine antagonists, drugs for the treatment of hyperlipoproteinemia, hypnotics and sedatives, immunosupressive agents, laxatives, methylxanthines, moncamine oxidase inhibitors, neuromuscular blocking agents, organic nitrates, opiod analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. It is further noted that electrolytes, or other ingredients that can be held or solubilized in a composition that can be incorporated into a matrix in a reservoir can also be delivered by the technique of the present invention.

The present invention is also useful in the controlled delivery of peptides, polypeptides, proteins and other such species. These substances typically have a molecular weight of at least about 300 daltons, and more typically have a molecular weight of about 300 to 40,000 daltons. Specific examples of peptides and proteins in this size range include, without limitation, luteinizing hormone-releasing hormone (LHRH), LHRH analogs such as goserelin, buserelin, gonadorelin, napharelin and leuprolide, growth hormone-releasing hormone (GHRH), growth hormone releasing factors (GHRF), GHRF fragments, insulin, insultropin, calcitonin, octreotide, endorphin, thyrotropin-releasing hormone (TRH), NT-36 (chemical name: [[(s)-4-oxo-2-azetidinyl]carbonyl]-L-histidyl-L-prolinamide), liprecin, pituitary hormones (e.g. human growth hormone (HGH), human menopausal gonadotropins (HMG), desmopressin acetate, etc), follicle luteoids, alpha atrial natriuretic factor (α-ANF), growth factors such as growth factor releasing factor (GFRF), beta-melanocyte-stimulating hormone (β-MSH, somatostatin, bradykinin, somatotropin, platelet-derived growth factor, asparaginase, bleomycin sulfate, chymopapain, cholecystokinin, chorionic gonadotropin, corticotropin (ACTH), erythropoietin, epoprostenol (platelet aggregation inhibitor), glucagon, human chorionic gonadotropin (HCG), hirulog, hyaluronidase, interferon, interleukins, menotropins (urofollitropin ( follicle stimulating hormone {FSH}) and luteinizing hormone, LH), oxytocin, streptokinase, tissue plasminogen activator, urokinase, vasopressin, desmopressin, adrenocorticotropic hormone (ACTH) analogs, atrial natriuretic peptide (ANP), ANP clearance inhibitors, angiotensin II antagonists, antidiuretic hormone agonists, bradykinin antagonists, cluster designation 4 (CD4), ceredase, enkephalins, Fab fragments, immunoglobulin E (IgE) peptide suppressors, insulin-like growth factor-1(IGF-1), neurotrophic factors, colony stimulating factors, parathyroid hormone and agonists, parathyroid hormone antagonists, fragments of parathyroid hormone, prostaglandin antagonists, pentigetide, protein C, protein S, renin inhibitors, thymosin alpha-1, thrombolytics, tissue necrosis factor-alpha (TNF-α), vaccines, vasopressin antagonists analogs, alpha-1 antitrypsin (recombinant), and tissue growth factor-beta (TGF-β).

In use, the trans-body-surface drug delivery device is applied on the body surface for therapeutically effective contact. When thermally controlled drug delivery is needed, the circuitry 124 can be activated to induce a temperature change (for example, an increase, in the case of a hydrogel with normal thermal sensitivity) on the thermoeffector 120 that includes thermoelectric couples, thereby causing the matrix in the drug reservoir 108 to shrink. As a result, the matrix decreases in its capacity to hold the drug solution that is in the drug reservoir 108 and makes available more of the drug solution to the body surface 112. After a desired amount of drug has been delivered, the heat flux of the thermoeffector surface facing the matrix to the drug reservoir can either be stopped or reversed. The reversal can be achieved by reversing the current flow through the couples in the thermoelectric device. By reversing the current flow, temperature change is reversed and the matrix is made to swell, thereby increasing the capacity to absorb more fluid. Thus, the heating and cooling of the matrix is actively reversibly controlled. In this way, any drug solution left on the body surface can be quickly absorbed from the body surface, thus dramatically reducing or even preventing the flux of the drug to the body surface from then on.

If desired, the temperature of the Peltier device can be controlled to vary in a pulsatile manner to modulate the drug flux in a pulsatile manner. If desired, after a period of heat flux, the current through the couples can simply be turned off, instead of being reversed, to slow the drug flux. Further, if desired, the temperature can be maintained at a steady level over a period of time by periodic modulation, e.g. by pulsatile heating and cooling, or intermittently turning off heating and cooling. Fine tuning of heating and cooling can be done by feedback control with measurement of the temperature at an appropriate location, e.g. in the drug reservoir. Also, temperature responsive drug delivery can be controlled by timing, i.e. by reversing or stopping the Peltier device after a set period of time has passed.

The temperature change that is applicable for controlling the thermal response of the matrix at the matrix proximate, the Peltier device can be about 25 to 60° C. for heating, 0 to 25° C. for cooling, more preferably about 25 to 44° C. for heating, 4 to 25° C. for cooling. Due to the sensitivity of the body tissue and the electronic components for electrotransport delivery, even more preferably the temperature change at the matrix proximate the Peltier device is about 25 to 45° C. for heating, 10 to 25° C. for cooling. It is to be understood that this temperature can be adjusted to effect desirable control based on data on the overall temperature of the matrix. Accordingly, the temperature at the Peltier device proximate the matrix can be about 25 to 45° C. for heating, 4 to 25° C. for cooling; more preferably about 25 to 45° C. for heating, 10 to 25° C. for cooling.

For a trans-body-surface drug delivery device of the present invention that includes also electrotransport, the thermally induced shrinking of the matrix can take place independently or simultaneously with the electrotransport. For example, in the case of a matrix with normal temperature sensitivity, a thermoelectric device can be used to heat the drug reservoir as the electrode provides a current to drive ionizable drug through the body surface. The heat will shrink the matrix, making more drug composition available to the body surface, as well as causing faster ion transport from the drug composition through the body surface.

As stated earlier, the present thermally controlled drug delivery can be adapted to use on traditional passive trandermal drug delivery patches and active eletrotransport devices such as iontophoretic devices. FIG. 4 illustrates schematically in portion how an electrotransport device, such as iontophoretic device can be adapted to have thermal control. The electrotransport device of FIG. 4 includes a thermoeffector 120 similar to the one shown in FIGS. 1 and 2. The electrical connection to the thermoeffector 120 and the thermoconductive seal (which can be optional) are not shown in the figure for clarity of illustration. An electrode 138 contacts the drug reservoir 108 (having a matrix 106) for providing a current to drive ionizable drug(s) through the body surface 112 of tissue 114, such as in transdermal delivery through skin. The electrode is connected to control circuitry 140 that is connected to ground 142 on the body of the patient for controlling the delivery of drug(s). Thermoconductive seal can be used to provide effective thermoconductive contact between the electrode 138 and the drug reservoir 108 as well to thermoeffector 120.

The electronics of the control 140 for electrotransport and the electronics for controlling the thermal modulation can be separate or can be integrated together in the same package by one skilled in the art. Further, it is not necessary that the thermoeffector 120 be on top of the electrode 138. Given enough room, as in a large drug reservoir, the two can be arranged as strips or pats side by side to provide the current, as well as the heating and cooling to effect control of drug delivery.

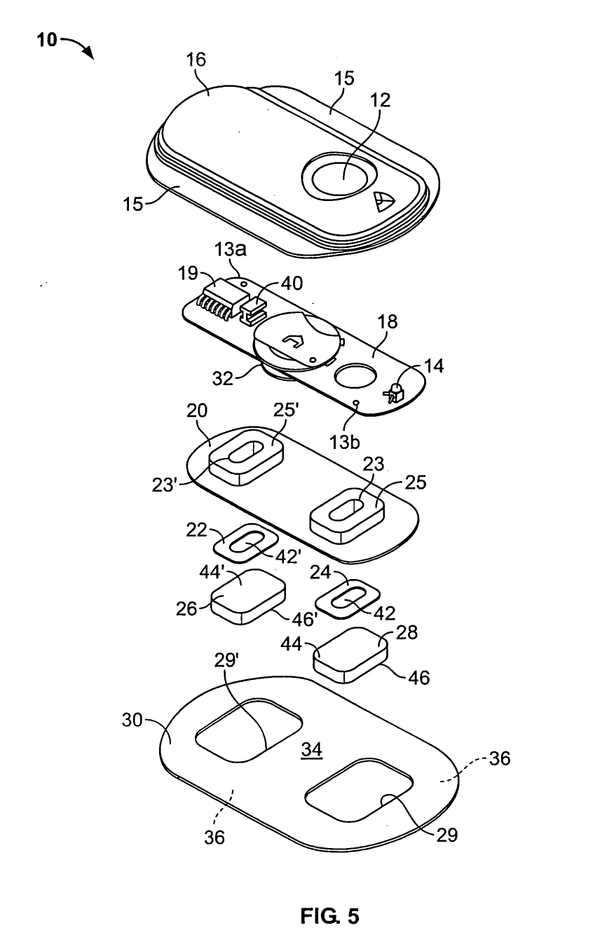

Electrotransport devices, such as iontophoretic devices are known in the art, e.g. U.S. Pat. No. 6,216,033, and can be adapted to function with the thermal control of the present invention as described above. A typical iontophoretic transdermal device that can be so adapted is described in the following. FIG. 5 depicts an exemplary electrotransport device that can be used in accordance with the present invention. FIG. 5 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable elastomer (e.g. ethylene vinyl acetate).

Printed circuit board assembly 18 comprises an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, which is preferably a button cell battery and most preferably a lithium cell. Other types of batteries may also be employed to power device 10.

The circuit outputs (not shown in FIG. 5) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′,29 in adhesive 30.

Upon depression of push button switch 12, the electronic circuitry on circuit board assembly 18 delivers a predetermined DC current to the electrodes/reservoirs 22,26 and 24,28 for a delivery interval of predetermined length, e.g. about 10-20 minutes. Preferably, the device transmits to the user a visual and/or audible confirmation of the onset of the drug delivery, or bolus, interval by means of LED 14 becoming lit and/or an audible sound signal from, e.g. a “beeper”. Drug, e.g. fentanyl or sufentanil, is then delivered through the patient's skin, e.g. on the arm, for the predetermined delivery interval. In practice, a user receives feedback as to the onset of the drug delivery interval by visual (LED 14 becomes lit) and/or audible signals (a beep from a “beeper”).

Anodic electrode 22 (preferably made of silver) and cathodic electrode 24 (preferably contains carbon and silver chloride) are loaded in a polymer matrix material. Both reservoirs 26 and 28 are preferably composed of polymer hydrogel materials as described herein. Electrodes 22, 24 and reservoirs 26, 28 are retained by lower housing 20. For cationic drugs, e.g. the anodic reservoir 26, is the “donor” reservoir that contains the drug and the cathodic reservoir 28 contains a biocompatible electrolyte, and optionally a second drug (anionic) to be delivered or an antimicrobial agent. If the electrode material is composed of materials that may undesirably absorb an ion, an ion exchange membrane can be located between the electrode 24 and the reservoir 28. Thus, for instance, an anion exchange membrane (not shown in FIG. 5, can be located between the cathodic electrode 24 and the cathodic reservoir 28 so that the cations will not penetrate through such membrane and therefore will not contact the cathodic electrode.

The push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is composed of polymeric sheet material that can be easily molded to form depressions 25,25′ and cut to form openings 23,23′. The lower housing, particularly the portions containing anodic reservoir 26 and cathodic reservoir 28, is composed of a polymeric material. The polymeric material is compatible with chemical agents in the reservoir so that the agents are not substantially absorbed into the polymeric material. Suitable polymeric materials include polyethylene terephthalate, polyethylene terephthalate modified with cyclohexane dimethylol (referred to as polyethylene terephthalate glycol or PETG) that renders the polymer more amorphous, polypropylene and mixtures thereof. Preferred polymeric materials are polyethylene terephthalate and PETG, which are both commercially available, and PETG is more preferred. A suitable PETG is available from Eastman Chemical Products, Inc. under the designation KODAR-PETG copolyester 6763.

The assembled device 10 is preferably water resistant (i.e. splash proof and is most preferably waterproof). The system has a low profile that easily conforms to the body thereby allowing freedom of movement at, and around, the wearing site. The anodic drug reservoir 26 and the cathodic reservoir 28 are located on the skin-contacting side of device 10 and are sufficiently separated to prevent accidental electrical shorting during normal handling and use.

The device 10 adheres to the patient's body surface (e.g. skin) by means of a peripheral adhesive 30 that has upper side 34 and body-contacting side 36. The adhesive side 36 has adhesive properties which assures that the device 10 remains in place on the body during normal user activity, and yet permits reasonable removal after the predetermined (e.g. 24 hour) wear period. Upper adhesive side 34 adheres to lower housing 20 and retains the electrodes and drug reservoirs within housing depressions 25, 25′ as well as retains lower housing 20 attached to upper housing 16. The device is also usually provided with a release liner (not shown) that is initially attached to body-contacting side 36 of adhesive 30 and removed prior to attachment to the patient. The release liner is typically siliconized polyethylene ethylene.

The push button switch 12 is located on the top side of device 10 and is easily actuated through clothing. A double press of the push button switch 12 within a short period of time, e.g. three seconds, is preferably used to activate the device 10 for delivery of drug, thereby minimizing the likelihood of inadvertent actuation of the device 10.

Upon switch activation an audible alarm signals the start of drug delivery, at which time the circuit supplies a predetermined level of DC current to the electrodes/reservoirs for a predetermined (e.g. 10 minute) delivery interval. The LED 14 remains “on” throughout the delivery interval indicating that the device 10 is in an active drug delivery mode. The battery preferably has sufficient capacity to continuously power the device 10 at the predetermined level of DC current for the entire (e.g. 24 hour) wearing period. The integrated circuit 19 can be designed so that a predetermined amount of drug is delivered to a patient over a predetermined time and then ceases to operate until the switch is activated again and that after a predetermined number of doses has been administered, no further delivery is possible despite the presence of additional drug in the donor reservoir.

As indicated above, suitable polymeric materials that can be used to form the cathodic reservoir wall include polyethylene terephthalate, polyethylene terephthalate modified with cyclohexane dimethylol, polypropylene and mixtures thereof. Preferably, the material is polyethylene terephthalate or polyethylene terephthalate modified with cyclohexane dimethylol. The polymeric materials can be formed into the desired shape (e.g. the form of the lower housing) by hot molding or any other suitable technique. Thermosensitive matrix such as thermosensitive hydrogel is contained in the reservoir, and a Peltier device can be used to conrol the drug delivery therefrom.

The aqueous medium to be contained in the anodic reservoir can be prepared in accordance with any conventional technique. For instance, when the aqueous medium is a hydrogel formulation, it can be composed of from about 10 to about 30% by weight of hydrophilic polymeric material, from about 0.1 to about 0.4% by weight of buffer, and the desired amount of drugs. The remainder is water and other conventional ingredients.

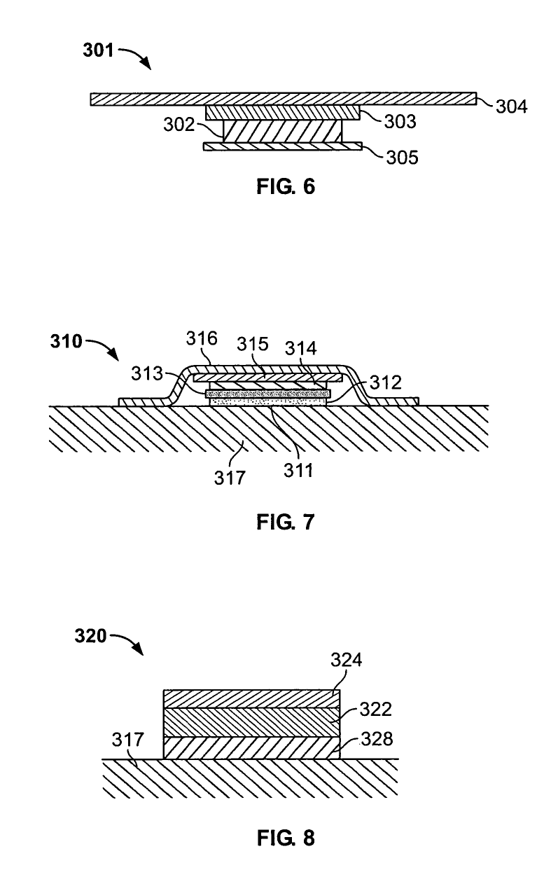

As stated above, traditional transdermal drug delivery patches, as in, e.g., U.S. Pat. No. 5,512,292, can be adapted to include thermal control according to the present invention. Such a typical transdermal drug delivery patch is described in the following. One embodiment of a transdermal delivery device of the present invention is illustrated in FIG. 6. In FIG. 6, device 301 is comprised of a drug-and permeation enhancer-containing reservoir (“drug reservoir”) 302 which is preferably in the form of a matrix containing the drug and the enhancer dispersed therein. A backing layer 303 is provided adjacent one surface of drug reservoir 302. Adhesive overlay 4 maintains the device 301 on the skin and may be fabricated together with, or provided separately from, the remaining elements of the device. With certain formulations, the adhesive overlay 304 may be preferable to an in-line contact adhesive, such as adhesive layer 328 as shown in FIG. 8. Backing layer 303 is preferably slightly larger than drug reservoir 302, and in this manner prevents the materials in drug reservoir 302 from adversely interacting with the adhesive in overlay 304. Reservoir 302 may be either saturated, unsaturated, or contain an amount of drug in excess of saturation. A strippable or removable liner 305 is also provided with device 301 and is removed just prior to application of device 301 to the skin.

FIG. 7 illustrates another embodiment of the invention, device 310, shown in placement on the skin 317. In this embodiment, the transdermal delivery device 310 comprises a multi-laminate drug formulation/enhancer reservoir 311 having at least two zones 312 and 314. Zone 312 consists of a drug reservoir substantially as described with respect to FIG. 6. Zone 314 comprises a permeation enhancer reservoir which is preferably made from substantially the same matrix as is used to form zone 312. Zone 314 comprises the permeation enhancer dispersed throughout, preferably in excess of saturation. A rate-controlling membrane 313 for controlling the release rate of the permeation enhancer from zone 314 to zone 312 is placed between the two zones. A rate-controlling membrane (not shown) for controlling the release rate of the enhancer and/or drug from zone 312 to the skin may also optionally be utilized and would be present between the skin 317 and zone 312.

The rate-controlling membrane may be fabricated from permeable, semipermeable or microporous materials which are known in the art to control the rate of agents into and out of delivery devices and having a permeability to the permeation enhancer lower than that of zone 312. Suitable materials include, but are not limited to, polyethylene, polyvinyl acetate and ethylene vinyl acetate copolymers.

Superimposed over the drug formulation/enhancer-reservoir 311 of device 310 is a backing 315 and an adhesive overlay 316 as described above with respect to FIG. 6. In addition, a strippable liner (not shown) would preferably be provided on the device prior to use as described with respect to FIG. 6 and removed prior to application of the device 310 to the skin 317.

In the embodiments of FIGS. 6 and 7, the carrier or matrix material has sufficient viscosity to maintain its shape without oozing or flowing. If, however, the matrix or carrier is a low viscosity flowable material, the composition can be fully enclosed in a dense non-porous or microporous skin-contacting membrane, as known to the art from U.S. Pat. No.4,379,454, for example.

An example of a presently preferred transdermal delivery device is illustrated in FIG. 8. In FIG. 8, transdermal delivery device 320 comprises a drug reservoir 322 containing together the drug and the permeation enhancer. Reservoir 322 is preferably in the form of a matrix containing the drug and the enhancer dispersed therein. Reservoir 322 is sandwiched between a backing layer 324, which is impermeable to both the drug and the enhancer, and an in-line contact adhesive layer 328. In FIG. 8, the drug reservoir 322 is formed of a material, such as a rubbery polymer, that is sufficiently viscous to maintain its shape. The device 320 adheres to the surface of the skin 317 by means of the contact adhesive layer 328. The adhesive for layer 328 should be chosen so that it is compatible and does not interact with any of the drug or, in particular, the permeation enhancer. The adhesive layer 328 may optionally contain the permeation enhancer and/or drug. A strippable liner (not shown) is normally provided along the exposed surface of adhesive layer 328 and is removed prior to application of device 320 to the skin 317. In an alternative embodiment, a rate-controlling membrane (not shown) is present and the drug reservoir 322 is sandwiched between backing layer 324 and the rate-controlling membrane, with adhesive layer 328 present on the skin-facing side of the rate-controlling membrane.

Various materials suited for the fabrication of the various layers of the transdermal devices of the above figures are known in the art or are disclosed in the aforementioned transdermal device patents previously incorporated herein by reference.

The matrix making up the drug reservoir can be a gel or a polymer. Suitable materials should be compatible with the drug and enhancer and any other components in the system. The matrix may be aqueous or non-aqueous based so long as the matrix can be operated according to the present invention. Aqueous formulations typically comprise water or water/ethanol and about 1-90 wt %, more preferably about 1-40 wt %, of a gelling agent that may or may not be thermosensitive, examples being xyloglucans, hydroxyethylcellulose, hydroxypropylcellulose, poly(N-isopropylacrylamide), poly(N-isopropylacrylamide) acrylamide copolymer, or others listed above. When using aqueous-based formulations, it is preferable to maintain the pH at a value that will maintain adequate stability of the active drug or to maintain appropriate thermosensitive gelation characteristics of the hydrogel. A thermoeffector can be used to contact the drug reservoir to control the thermosensitive matrix for effective drug delivery.

Permeation enhancers can be used for increasing the skin permeability of the drug or drugs to achieve delivery at therapeutically effective rates. Such permeation enhancers can be applied the skin by pretreatment or currently with the drug, for example, by incorporation in the reservoir. A permeation enhancer should have the ability to enhance the permeability of the skin for one, or more drugs or other biologically active agents. A useful permeation enhancer would enhance permeability of the desired drug or biologically active agent at a rate adequate for therapeutic level from a reasonably sized patch (e.g. about 5 to 50 cm²). Permeation enhancers should be compatible with a drug must not adversely interact with the adhesive of the in-line contact adhesive layer if one is present. Examples of permeation enhancers are disclosed in previous ALZA patents cited and previously incorporated by reference and can be selected from, but are not limited to, fatty acids, monoglycerides of fatty acids such as glycerol monolaurate, glycerol monooleate, glycerol monocaprate, glycerol monocaprylate, or glycerol monolinoleate; lactate esters of fatty acids such as lauryl lactate, cetyl lactate, and myristyl lactate; acyl lactylates such as caproyl lactylic acid; esters of fatty acids having from about 10 to about 20 carbon atoms, including, but not limited to, isopropyl myristate, and ethyl palmitate; alkyl laurates such as methyl laurate; dimethyl lauramide; lauryl acetate; monoalkyl ethers of polyethyleneglycol and their alkyl or aryl carboxylic acid esters and carboxymethyl ethers such as polyethylene glycol-4 lauryl ether (Laureth-4) and polyethylene glycol-2 lauryl ether (Laureth-2); polyethylene glycol monolaurate; myristyl sarcosine; Myreth-3; and lower C₁₋₄ alcohols such as isopropanol and ethanol, alone or in combinations of one or more.

A preferred permeation enhancer according to this invention comprises a monoglyceride of a fatty acid together with a suitable cosolvent, including, but not limited to, lauryl acetate as disclosed in WO 96/40259 and esters of C₁₀-C₂₀ fatty acids such as lauryl lactate, ethyl palmitate, and methyl laurate. Ethyl palmitate has been found to be particularly desirable as it is obtainable at a high degree of purity, thus providing a purer and better defined permeation enhancer and a system that is more readily characterized. According to a particularly preferred embodiment, the permeation enhancer comprises glycerol monolaurate (GML) and ethyl palmitate within the range of 1-25 wt % and 1-20 wt %, respectively, at a ratio of GML/ethyl palmitate within the range of 0.5-5.0, preferably 1.0-3.5. A particularly preferred embodiment comprises 20 wt % GML and 12 wt % ethyl palmitate.

The permeation-enhancing mixture is dispersed through the matrix or carrier, preferably at a concentration sufficient to provide permeation-enhancing amounts of enhancer in the reservoir throughout the anticipated administration period. Where there is an additional, separate permeation enhancer matrix layer as well, as in FIGS. 3 and 4, the permeation enhancer normally is present in the separate reservoir in excess of saturation.

The amounts of the drug that are present in the therapeutic device, and that are required to achieve a therapeutic effect, depend on many factors, such as the minimum necessary dosage of the particular drug; the permeability of the matrix, of the adhesive layer and of the rate-controlling membrane, if present; and the period of time for which the device will be fixed to the skin. There is, in fact, no upper limit to the maximum amounts of drug present in the device. The minimum amount of each drug is determined by the requirement that sufficient quantities of drug must be present in the device to maintain the desired rate of release over the given period of application.

If desired, the drug can be dispersed through the matrix at a concentration in excess of saturation in order to maintain unit activity throughout the administration period. The amount of excess is determined by the intended useful life of the system. However, the drug may be present at initial levels below saturation without departing from this invention. Generally, the drug may be present at initially subsaturated levels when: 1) the skin flux of the drug is sufficiently low such that the reservoir drug depletion is slow and small; 2) non-constant delivery of the drug is desired or acceptable; and/or 3) saturation or supersaturation of the reservoir is achieved in use by cosolvent effects which change the solubility of the drug in use such as by loss of a cosolvent or by migration of water into the reservoir.

In the present invention, the drug is delivered through the skin or other body surface at a therapeutically effective rate (that is, a rate that provides an effective therapeutic result) and the permeation enhancer is delivered at a permeation-enhancing rate (that is, a rate that provides increased permeability of the application site to the drug) for a predetermined time period.

A preferred embodiment of the present invention is a multilaminate such as that illustrated in FIG. 8 (either with or without a rate-controlling membrane) wherein reservoir 22 comprises, by weight, 1 to 90% polymer (preferably 40%), 0.01-40% drug, and 1-70% of one or more permeation enhancer. The in-line adhesive layer 28 contains an adhesive that is compatible with the permeation enhancer. In another preferred embodiment of the invention, a multilaminate such as that in FIG. 8 includes reservoir 22 comprising, by weight, 5 to 90% polymer (preferably 20%), 0.01-40% drug, 1-70% of one or more permeation enhancer.

The devices of this invention can be designed to effectively deliver a drug for an extended time period of up to 7 days or longer. Seven days is generally the maximum time limit for application of a single device because the body surface (e.g. skin) site may be affected by a period of occlusion greater than 7 days, or other problems such as the system or edges of the system lifting off of the skin may be encountered over such long periods of application. Where it is desired to have drug delivery for greater than 7 days (such as, for example, when a hormone is being applied for a contraceptive effect), when one device has been in place on the skin for its effective time period, it is replaced with a fresh device, preferably on a different skin site.

### EXAMPLES

### Example 1

### Hydrogel Preparation

Poly(N-isopropylacrylamide) (PNIPA) gel can be synthesized by the free radical solution copolymerization/crosslinking of PNIPA monomer. Approximately 9.6 g of NIPA monomer per 0.4 g of the crosslinker N,N′-methylenebisacrylamide can be dissolved in 100 mL distilled water. Reagent grade ammonium persulfate (“APS”) can be used to initiate the reaction and reagent grade N,N,N′,N′- tetramethylethylenediamine (“TEMED”) can be added as an accelerator. Freshly prepared initiator solutions are to be added to the solution to result in concentrations of 0.30 mg of APS per mL of monomer solution, and 0.15 mg of TEMED per mL of monomer solution. All solutions are degassed under 24 inches Hg of vacuum for approximately 15 minutes. The gels are synthesized in a glove box under a nitrogen atmosphere containing less than 2% oxygen. The initiators can be added to the monomer solution and the solution degassed under vacuum while stirring on a magnetic stirrer for 10-15 minutes. Bonded gel membranes are made by casting gel solutions between glass plates separated by a high purity silicone rubber gasket, tubes, or rectangular pipes. An impermeable plastic substrate (GELBOND® polyacrylamide support medium manufactured by FMC BioProducts, Rockland Me.) having a thickness of approximately 0.2-0.6 mm can be placed on one inside surface of the glass plate prior to gel casting. Gelation typically occurs within 1-2 hours, after which the molds can be removed from the glove box and placed in a refrigerator at 32° C. for 24 hours to allow the reaction to approach completion. The resulting PNIPA gels can have thicknesses ranging from about 0.2 mm to 1 mm, when swollen in 25° C. water or other aqueous solvents. After casting, the membrane samples are soaked in distilled water for approximately 72 hours to remove any unreacted compounds.

### Example 2

### Passive Drug Delivery by Diffusion

The present invention can be used to administer a drug transdermally that otherwise would have a diffusion coefficient or a permeability coefficient across a rate limiting membrane that would be inadequate. A user places a patch with a heat-modulatable matrix containing a drug onto the skin of the patient and uses a Peltier device attached to the matrix to modulate the reversible heating characteristics to facilitate increased absorption of the drug. Increased temperature provided by the Peltier device, for example, increases the diffusion coefficient of the active ingredient in the formulation and/or increases the permeability coefficient of the drug across the rate limiting membrane of patch and subsequently through the skin. The rate at which the active ingredient enters the body would thereby also increase and in turn, increase the concentration of the active ingredient in the patient's blood stream. When sufficient levels of the active ingredient are attained in the blood stream, the user or patient can turn-off the delivery of drug by cooling the patch with the Peltier device.

### Example 3

### Passive Drug Delivery by Diffusion

The present invention can be used to administer a drug transdermally in a reversible, as-needed basis by a user or patient. Such a case would require intermittent delivery into the blood stream through the skin. A user places a patch with a hydrogel containing a drug onto the skin of the patient and controls the reversible transdermal flux and delivery by either heating or cooling a thermosensitive hydrogel matrix containing the drug. Increased temperature provided by the Peltier element causes the hydrogel to shrink and thus release free drug from the matrix, making the drug more available for flux across the rate-limiting membrane and subsequently skin. A higher temperature also increases the diffusion rate across the skin. When the user needs to turn off or stop further delivery of the drug, the Peltier element is controlled to cool the hydrogel matrix causing it to swell and thus re-absorb free drug and formulation. Thus, sufficient levels of the active ingredient can be attained in the blood stream by the user or patient on an as-needed basis by heating or cooling the patch with the Peltier element. Examples of drugs that would benefit from this invention are drugs subject to craving such as nicotine, pain medications for intermittent or breakthrough pain, anti-parkinson's disease agents to control motor complications, etc.

### Example 4

### Electrotransport (Active) Drug Delivery

A 2% fentanyl matrix is formulated in a poly(N-isopropylacrylamide)acrylamide copolymer (pNIPAA-AA) hydrogel. A 2 cm² film of thermosensitive hydrogel with a thickness of 20-30 mils (508 -676 microns) is die-cut and weighed. The film is placed in the donor housing of an iontophoretic transdermal drug delivery device, as in U.S. Pat. No. 6,216,033, and hydrated with 2.5 times their weight in drug solution as described above. The fentanyl solutions can be prepared with sufficient fentanyl HCl to yield a final drug concentration in the hydrogel of 2 wt %. The hydrogel matrix may also contain appropriate permeation enhancers as described above. Alternatively, the films of the hydrogel may be loaded with 2% fentanyl by saturation absorption as described above and then mounted into the donor hoursing of the iontophoretic transdermal drug delivery device. The anodic compartment (2 cm²) of the device can be be filled with 350-450 ml of the hydrocortisone gel with room to contain the swollen gel. The cathodic compartment (2 cm²) is filled with 350-450 ml of a sodium chloride pNIPAA-AA gel. The system and controller can be secured to the skin with appropriate adhesives. The controller can be turned on to deliver 200 MA/cm² with or without heating or cooling by the Peltier element. Transdermal flux can be initiated by using the Peltier element to heat the drug reservoir to above the LCST to de-swell or shrink the matrix and release drug from the matrix. Free drug in formulation can then be driven by iontophoresis and application of a suitable current per unit area. When delivery of fentanyl has to be turned off, the hydrogel matrix will cooled using the Peltier element to a temperature below the LCST causing the gel to swell and this re-absorb the fentanyl formulation and thus is unavailable for flux through the skin.

The entire disclosure of each patent, patent application, and publication cited or described in this document is hereby incorporated herein by reference. Embodiments of the present invention have been described with specificity. It is to be understood that various combinations and permutations of various parts and components of the schemes disclosed herein can be implemented by one skilled in the art without departing from the scope of the present invention. It is to be further understood that when an object or material is mentioned in an embodiment, a plurality or combination of the object or material is also contemplated as useful unless specified otherwise.

What is claimed is:

1. A device for the administration of a pharmaceutical agent to an individual at a therapeutically effective rate, by passing through a body surface, comprising: a reservoir including a pharmaceutical agent and a thermoeffector having a first surface proximate the reservoir, the first surface controllable to at least one of heating and cooling the reservoir to affect passage rate of the pharmaceutical agent through the body surface.

2. The device of claim 1 wherein the first surface faces the reservoir and is controllable to reversibly heat or cool.

3. The device of claim 1 wherein the thermoeffector can be controlled to reverse heat flow to the reservoir, thereby modulating delivery of the pharmaceutical agent from the reservoir.

4. The device of claim 1 wherein the first surface faces the reservoir and is controllable to reversibly heat or cool and the device has a second surface that heats while the first surface cools and cools while the first surface heats.

5. The device of claim 4 wherein the thermoeffector includes a plate having the first surface proximate the reservoir and having the second surface facing away from the reservoir, the plate having a thickness less than a thickness of the reservoir.

6. The device of claim 5 wherein the thermoeffector plate includes multiple layers, at least one of which includes semiconductor material having a junction that can either heat or cool depending on direction of electrical current passing therethrough.

7. The device of claim 6 wherein the thermoeffector plate includes two insulation layers with an array of Bismuth-containing semiconductor elements positioned therebetween.

8. The device of claim 7 wherein the thermoeffector plate has a thickness of less than 4 mm.

9. The device of claim 7 wherein the thermoeffector plate has a thickness of between 1 mm and 4 mm.

10. The device of claim 6 comprising a feedback control for controlling the temperature of the reservoir.

11. The device of claim 6 further comprising a matrix having heat sensitive polymer in the reservoir, the heat sensitive polymer causing the matrix to change size by swelling and shrinking as a function of temperature such that the matrix upon swelling increases the matrix capacity to hold liquid and upon shrinking decreases capacity to hold liquid.

12. The device of claim 11 further comprising hydrogel in the reservoir, the hydrogel being adapted to shrink as the temperature falls and swell as the temperature rises.

13. The device of claim 11 further comprising hydrogel in the reservoir, the hydrogel being adapted to shrink as the temperature rises and swell as the temperature falls.

14. The device of claim 11 wherein the hydrogel contains a polymer selected from a group consisting of poly(N-isopropylacrylamide), copolymer containing poly(N-isopropylacrylamide), and polypeptide-containing polymers.

15. The device of claim 11 wherein the hydrogel contains a polymer selected from a group consisting of poly(N-isopropylacrylamide) homopolymer, poly(N-isopropylacrylamide)acrylamide copolymer, copolymer of poly(N-isopropylacrylamide) containing silane monomers selected from [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide), dicarboxymethylaminopropyl methacrylamide with protein moieties, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene)derivatives.

16. The device of claim 11 comprising circuitry that causes the matrix to swell and circuitry that causes the matrix to shrink.

17. The device of claim 10 comprising circuitry that causes the matrix to shrink and then swells after one of reaching a specific time and reaching a specified temperature at the reservoir.

18. The device of claim 10 wherein the device does not include a sharp object that punctures through the body surface into the tissue beneath the body surface.

19. The device of claim 4 further comprising electrode contacting the reservoir to provide an electrical potential to drive the pharmaceutical agent through the body surface by electrotransport.

20. A method for making a device for trans-body-surface delivery of pharmaceutical agent, comprising: forming a reservoir including a pharmaceutical agent and positioning a thermoeffector proximate to the reservoir, the thermoeffector having a first surface that is controllable to at least one of heating and cooling the reservoir to affect trans-body-surface passage rate of the pharmaceutical agent.

21. The method of claim 20 comprising positioning the first surface to the reservoir and wherein the thermoeffector is controllable to reversibly heat or cool and the device has a second surface that heats while the first surface cools and cools while the first surface heats.

22. The method of claim 21 wherein the thermoeffector includes a plate having the first and second surfaces and the method comprising placing the plate with the first surface proximate the reservoir and having the second surface facing away from the reservoir, the plate having a thickness less than a thickness of the reservoir.

23. The method of claim 22 wherein the plate includes semiconductor material having a junction that can either heat or cool depending on direction of electrical current passing therethrough, and the method includes positioning an array of Bismuth containing semiconductor elements between two insulation layers.

24. The method of claim 23 further comprising including a matrix in the reservoir, the matrix having thermosensitive polymer that causes the matrix to swell and shrink as a function of temperature.

25. The method of claim 20 further comprising including hydrogel in the reservoir, the hydrogel being adapted to one of shrinking and swelling as the temperature falls and reversing the shrinking or swelling as the temperature rises.

26. The method of claim 20 comprising including in the hydrogel a polymer selected from a group consisting of poly(N-isopropylacrylamide), copolymer containing poly(N-isopropylacrylamide), and polypeptide containing polymers.

27. The method of claim 20 comprising including in the hydrogel a polymer selected from a group consisting of poly(N-isopropylacrylamide)homopolymer, poly(N-isopropylacrylamide)acrylamide copolymer, copolymer of poly(N-isopropylacrylamide) containing silane monomers including [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide) and dicarboxymethylaminopropyl methacrylamide with protein moieties, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene) derivatives.

28. The method of claim 20 comprising including a heat sensitive polymer in the reservoir such that the matrix upon cooling swells to increase the matrix capacity to hold liquid and upon heating shrinks to decrease capacity to hold liquid.

29. The method of claim 21 further comprising placing an electrode at the reservoir to provide an electrical potential to drive the pharmaceutical agent through the body surface by electrotransport.

30. A method for trans-body-surface delivery of pharmaceutical agent, comprising: providing a reservoir including a pharmaceutical agent and actively reversibly heating the reservoir to affect passage rate of the pharmaceutical agent.

31. The method of claim 30, wherein the thermoeffector has a first surface and a second surface, and comprising positioning the first surface of thermoeffector thermoconductively proximate to the reservoir and effect a temperature change on the first surface while effecting a temperature change on the second surface opposite to that on the first surface.

32. The method of claim 30 wherein the thermoeffector includes semiconductor material having a junction that can either heat or cool depending on direction of electrical current passing therethrough, and the method includes passing a current through an array of Bismuth-containing semiconductor elements that are positioned between two insulation plates.

33. The method of claim 31 further comprising swelling and shrinking a hydrogel in the reservoir as a fiction of temperature.

34. The method of claim 31 further comprising swelling and shrinking a hydrogel in the reservoir, the hydrogel shrinks as the temperature rises and swells as the temperature falls.

35. The method of claim 31 comprising including swelling and shrinking a hydrogel in the reservoir, the hydrogel including a polymer selected from a group consisting of poly(N-isopropylacrylamide), copolymer containing poly(N-isopropylacrylamide), and polypeptide-containing polymers.

36. The method of claim 30 comprising electrically causing the matrix to shrink to provide liquid on the body surface and then electrically causing the matrix to swell to absorb liquid from the body surface.

37. The method of claim 30 comprising delivering the pharmaceutical agent without using a sharp object that punctures through the body surface into the tissue beneath the body surface.

38. The method of claim 30 further providing an electrical potential to drive the pharmaceutical agent through the body surface by electrotransport.

39. A device for the administration a liquid to an object surface, comprising: a liquid in a matrix and a thermoeffector having a first surface that is actively reversibly controllable to heat and controllable to cool to cause the matrix to change volume, thereby controlling the amount of liquid being deposited on the body surface.

40. The device of claim 39 wherein the first surface faces the matrix and is controllable to reversibly heat or cool, whereby heating causes the matrix to shrink and cooling causes the matrix to swell.

41. The device of claim 39 wherein the thermoeffector has a plate including multiple layers, at least one of which includes semiconductor material having a junction that can either heat or cool depending on direction of electrical current passing therethrough.

42. The device of claim 39 wherein the thermoeffector has a plate that includes two insulation layers with an array of Bismuth-containing semiconductor elements positioned therebetween.

43. The device of claim 40 comprising circuitry that causes heating the matrix by heat conduction to cause the matrix to shrink and circuitry that cools the matrix by heat conduction to cause the matrix to swell.

44. The device of claim 30 comprising circuitry that causes the matrix to shrink and then after one of reaching a specific time and reaching a specified temperature at the reservoir causes the matrix to swell.

45. A device for the administration of a pharmaceutical agent to an individual at a therapeutically effective rate, by passing through a body surface, comprising: a matrix including a pharmaceutical agent and a thermoeffector having a first surface proximate the matrix, the first surface controllable to reversibly heat and cool the matrix to affect passage rate of the pharmaceutical agent through the body surface, the thermoeffector having a second surface that heats while the first surface cools and cools while the first surface heats, the thermoeffector having multiple layers including at least one layer having Bismuth-containing semiconductor material with a junction that can either heat or cool depending on the direction of electrical current passing through, wherein the heating and cooling of the matrix changes the matrix's capacity to hold a liquid containing the pharmaceutical agent.

46. A method of using of a composition comprising a drug, together with a carrier, the composition releasing the drug in a controllable dose over time, the carrier having a reservoir containing the composition and having a thermoeffector proximate the reservoir, comprising controlling the thermoeffector for reversibly heating the reservoir to control release of the drug to deliver through a body surface of a patient.
